(19) United States
(12) Patent Application Publication
(10) Pub. No.: US 2005/0089893 A1
(43) Pub. Date: Apr. 28, 2005

(54) Methods and compositions for in vitro and in vivo use of parallel stranded hairpins and triplex structures as nucleic acid ligands

(76) Inventors: Martin J. Lopez, Vancouver (CA); Martin Munzer, Parkland, FL (US); Ramon Eritja, Barcelona (ES)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/912,032

(22) Filed: Aug. 4, 2004

Related U.S. Application Data
(60) Provisional application No. 60/493,092, filed on Aug. 6, 2003.

(51) Int. Cl. C12Q 1/68; C07H 21/02; C07K 14/00

(52) U.S. Cl. 435/6; 530/395; 536/23.1

(57) ABSTRACT

The invention sets forth parallel-stranded hairpins and parallel-stranded hairpins carrying a single strand target, the parallel-stranded hairpins containing 8-aminopurine residues and are able to bind a target molecule of interest due to specificity in the two-dimensional and three-dimensional structures of the parallel-stranded hairpins and parallel-stranded hairpins carrying a single strand target. The invention also relates to creation of a library of parallel-stranded hairpins and associated triplexes for use as aptamers, as well as methods of synthesis of parallel-stranded hairpins and use of the structures of the invention to detect and eliminate molecules of interest.

### CLAIM TO PRIORITY

This application claims the benefit of priority of co-pending prior U.S. Provisional Patent Application No. 60/493,092, filed Aug. 6, 2003, entitled “Methods And Compositions For In Vitro And In Vivo Use Of Parallel Stranded Hairpins And Triplexes Structures As Nucleic Acid Ligands,” and having the same inventors as set forth herein, namely Martin Lopez, Ramon Eritja, and Martin Munzer. That application is incorporated by reference as if fully rewritten herein.

### SEQUENCE IDENTIFICATION LISTING

This application includes sequence identification listings both on paper and in computer readable form. Applicants state that the sequence listing information recorded in computer readable form is identical to the written (on paper) sequence listing.

### FIELD OF THE INVENTION

The invention relates to novel oligomer analogs and their use as artificial nucleic acid ligands. More specifically, the invention involves parallel-stranded hairpins and parallel-stranded hairpins carrying a single strand target, wherein the parallel-stranded hairpins containing 8-aminopurine residues, and their ability to bind a target molecule of interest. The invention also relates to creation of a library of parallel-stranded hairpins and associated triplexes for use as aptamers, as well as methods of synthesis of parallel-stranded hairpins.

### BACKGROUND OF THE INVENTION

Drug development has traditionally focused on active sites of proteins, and on identifying molecules such as HIV protease inhibitors that bind to the active sites of proteins and block directly interactions with the natural substrate, as discussed in DeDecker B., “Allosteric drugs: thinking outside the active-site box,” Chem. & Biol. (2000) 7:R103-R107. Besides this direct mode of enzymatic regulation, nature makes extensive use of allosteric interactions to regulate the activities of proteins. These types of interactions allow much to be accomplished by a small molecule. One example of an allosteric interaction is tryptophan repression, wherein tryptophan (trp) amino acid, a small molecule, after binding to the trp repressor confers to the protein DNA binding properties, as reported in Santillan, M., and Mackey, M. “Dynamic regulation of the tryptophan operon: A modeling study and comparison with experimental data”, Proc. Nat'l Acad. Sci. USA (1998) 95:3077-3081. Consequently, binding of small molecules to any protein target may confer a different property to the protein that may affect the protein's interaction with specific substrates, receptors, or other structures of interest.

As reported by Hermann, T., and Patel, D. “Adaptive Recognition by Nucleic Acid Aptamers,” Science (2000) 287:820-825, and Morris, K., et al., “High affinity ligands from in vitro selection: Complex targets,” Proc. Nat'l Acad. Sci. USA (1998) 95:2902-2907, nucleic acid aptamers are artificial nucleic acid ligands, typically, but not exclusively, composed of RNA, single-stranded DNA or a combination of these, with properties for high affinity binding to given ligands. They are versatile tools that can greatly enhance the efficiency of modern drug development. Exhibiting binding characteristics comparable to or even better than monoclonal antibodies, these ligands can be used as detection probes, highly efficient inhibitors of protein function or specific competitors, as reported in Brody E., et al., “Aptamers as therapeutic and diagnostic agents,” Rev. in Mol. Biotech. (2000) 74:5-13; Hesselberth, J., et al., “In vitro selection of nucleic acids for diagnostic applications,” Rev. in Mol. Biotech. (2000) 74:15-25; O'Sullivan C., “Aptasensors-the future of biosensing?” Anal. Bioanal. Chem. (2002) 372:44-48.

As discussed in O'Sullivan, C., “Aptasensors-the future of biosensing?” Anal. Bioanal. Chem. (2002) 372:44-48, in 1990 the laboratories of Szostak, Gold and Joyce reported the in vitro selection of novel ligands from combinatorial nucleic acid libraries. In the SELEX® method (Systematic Evolution of Ligands by Exponential enrichment), an oligonucleotide library is synthesized wherein the oligonucleotide comprises 5′ and 3′ regions of defined sequence and a central region of random sequence, as reported in James, W., “Aptamers. Encyclopedia of Analytical Chemistry,” (2000) 1-23; Kusser, W., “Chemical modified nucleic acid aptamers for in vitro selections: evolving evolution,” Rev. in Mol. Biotech. (2000) 74:27-38; Osborne S., et al., “Nucleic Acid Selection and the Challenge of Combinatorial Chemistry,” Chem. Rev. (1997) 97:349-370; Ulrich, H., et al., “RNA and DNA aptamers as potential tools to prevent cell adhesion in disease,” Brazilian Journal of Medical and Biological Research (2001) 34:295-300. Following conversion into dsDNA by RT-PCR, products can be transcribed in vitro to provide a library of RNA sequences. If one consider the length of a randomized section to be n, then the library contains 4ⁿ different sequences. These sequences adopt characteristic three-dimensional structure as a result of sequence-determined intramolecular interactions. These structures may include, but are not limited to, formation of hairpin loops stabilized by a combination of Watson-Crick and non-canonical intramolecular interactions. Among all the DNAs/RNAs sequences tested, one may find one or more exhibiting a shape that due to its physico-chemical properties will bind to a selected target molecule.

As reported in U.S. Pat. No. 5,270,163, to Gold, et al., in the SELEX protocol affinity separation is done under neutral pH or higher, and because parallel duplexes with natural bases are only formed under acidic conditions, it would be very difficult to identify a parallel duplex as aptamer for a given protein or other kind of molecules using this method. In addition, parallel structures at neutral pH are only preferred in highly A-T rich sequences and long sequences. This parallel A-T duplex has a different structure (reversed Watson-Crick structures) compared with parallel stranded hairpins carrying 8-aminopurines (Hoogsteen structure) described by Aviño, A., et al., “Properties of triple helices formed by parallel-stranded hairpins containing 8-aminopruines,” Nucleic Acids Res. (2002) 30:2609-2619, and in U.S. patent application Ser. No. 10/690,274, “Triplex Forming Oligonucleotides Containing modified purines and their applications.” FIG. 1 shows hypothetical structures of parallel-strnded hairpin triplexes with 8-aminopurine substitutions. All these facts make parallel-stranded hairpins of the invention, described below, preferential structures for use as potential nucleic acid ligands.

Nucleic acid aptamers range in size from about 6 to about 40 kDa. They may have complex three-dimensional structures. Aptamers have been reported to bind amino acids, drugs, proteins, and other molecules. They have purportedly been used to analyze the natural process of nucleic acid-protein recognition, to generate inhibitors of enzymes, hormones, toxins, and to detect the presence of target molecules in complex mixtures, among other uses. They have been reported to bind to their targets with dissociation constant (Kd) typically in the low nanomolar range, and they can purportedly distinguish enantiomers of small molecules or minor sequence variants of macromolecules.

The desired property of an aptamer is typically the ability to bind a molecule of interest. Depending on the application, binding properties may be a fast association rate, slow dissociation rate, high affinity, low affinity to closely related molecules, or a combination of these. The property or properties are a function of the three-dimensional structure of the folded nucleic acid and are a combination of van der Waals surface contacts, hydrogen bonds, stacking interactions and other non-covalent bonds that can form between the aptamer and its target. The three-dimensional structure of an aptamer is uniquely determined by the sequence of its bases.

Nucleic acid aptamers have a broad field of applications, including diagnostic and therapeutic purposes. One example of a diagnostic aptamer application is a RNA aptamer said to be specific for S-adenosylhomocysteine (SAH), a potential diagnostic marker for cardiovascular disease, as reported in James, W., “Nucleic Acid and polypeptide aptamers: a powerful approach to ligand discovery,” Current Opinion in Pharmacology (2001) 1:540-546. SAH is known to be elevated in plasma or serum from patients having certain forms of cardiovascular disease. Therepeutic aptamers have also been described, including aptamer antagonists of the toxin Ricin. Also described are aptamers that are inhibitors of certain viral enzymes such as HIV-1 reverse transcriptase and the NS3 protease of hepatitis C virus, as reported in Kandimalla, E.R., et al., “DNA duplexes of 3′-3- and 5′-5′-linked oligonucleotides and triplex formation with RNA and DNA pyrimidine single strands: experimental and molecular modeling studies,” Biochem. (1996) 35:15332-15339.

The potential utility of aptamers as therapeutic agents is reportedly enhanced by chemical modifications of the nucleic acid oligonucleotides that lend resistance to nuclease attack. Example of modifications are the addition of phosphorothioates, or substitutions of the 2′-OH groups of pyrimidines with 2′-F, 2′-NH2, or 2′-Ome. Aptamers recognize epitopes with the same specificity as antibodies but in an aspect different from antibodies they posses low immunogenicity and are not subjected to proteolytic degradation.

The human genome contains more than 100,000 pyrimidine:purine tracts that are about 200 to about 300 bp in length. An interesting aspect of those sequences is that they can form triplexes, as discussed in Agazie, Y., et al., “Triplex DNA in the nucleus: direct binding of triplex-specific antibodies and their effect on transcription, replication, and cell growth,” Biochemistry J. (1996) 316:461-46. In 1991 Kiyama R. et al. reported a purified triplex DNA-binding protein from human cells, in Kiyama, R., “A triplex DNA-binding protein from human cells: Purification and characterization,” Proc. Nat'l Acad. Sci. USA (1991) 88:10450-10454. Involvement of triplexes in cell activity such as control of gene expression has been reported. For example, a transcription factor known as BP-8 is said to bind to a triplex in the promoter region of γ-globin gene as reported in Horwitz, E., et al., “A Human Protein containing a ‘Cold Shock’ domains binds specifically to H-DNA upstream from the Human γ-globin genes,” JBC (1994) 19:14130-14139.

It has been reported that oligonucleotides bind to homopurine-homopyrimidine sequences of double stranded DNA by forming triple helices. Güimil, R., et al. “Theoretical calculations, synthesis and base pairing properties of oligonucleotides containing 8-amino-2′-deoxyadenosine,” Nucleic Acids Res. (1999) 27:1991-1999. One of the problems for the development of applications based on triple helix formation is the low stability of triple helices, especially at neutral pH (physiological pH). To overcome this problem effort has been directed to design and preparation of modified oligonucleotides in order to enhance triple helix stability. The most studied type of triple helix formation is the so called purine:pyrimidine:pyrimidine motif (FIGS. 1 and 2). In this motif, the purine:pyrimidine strands correspond to the target double stranded DNA sequence (known as the Watson-Crick purine and pyrimidine strands), and the Hoogsteen strand is a pyrimidine strand used for the specific recognition of the double-stranded DNA, as reported in Soliva R., et al., “DNA-triplex stabilizing properties of 8-aminoguanine. Nucleic Acids Res 2000 28:4531-4539.

Most of the reported base analogues studied for triplex helix stabilization are modified pyrimidines located at the Hoogsteen strand. However, an alternative approach based on the use of parallel-stranded duplexes has been reported. In an exemplary parallel-stranded duplex, purine residues are linked to a pyrimidine chain of inverted polarity by 3′-3- or 5′-5′ intemucleotide junctions (FIG. 2). Such parallel-stranded DNA hairpins have reportedly been synthesized and are said to bind single-stranded DNA and RNA targets by triplex formation. Oligonucleotides containing 8-aminopurines may replace natural purines in triplexes. The introduction of an amino group at position 8 of the adenine, guanine, and hypoxanthine, increases the stability of triplex helix owing to the combined effect of the gain in one Hoogsteen purine-pyrimidine H-bond, and the ability of the amino group to be integrated into the ‘spine of hydration’ located in the minor-major groove of the triplex structure.

Interest in branched nucleic acids or nucleic acid dendrimers has grown recently, resulting in increased attention to synthesis of these structures. Branched oligoribonucleotides have been synthesized by solution-phase or solid-phase methods, as reported in Grøtli, M., et al., “Solid-phase synthesis of branched RNA and branched DNA/RNA chimeras,” Tetrahedron (1997) 53:11317-11346, and Kierzek, R., et al., “Chemical synthesis of branched RNA,” Nucleic Acids Res. (1986) 14:4751-4764. Moreover, branched nucleic acids have been purportedly prepared using nucleoside branching points other than the 2′ and 3′ positions of a ribonucleoside such as 4′-C-(hydroxymethyl)thymidine, as stated in von Büren, M., et al., “Branched Oligodeoxynucleotides: Automated Synthesis and Triple Helical Hybridization Studies,” Tetrahedron (1995) 51:8491-8506, and Thrane, H., et al., “Novel linear and branched oligodeoxynucleotide analogues containing 4′-C-(Hydroxymethyl) thymidine,” Tetrahedron 51:10389-10402. The complexity of the synthesis of the branching molecules and the low yields obtained has been triggered the used of non-nucleoside branching molecules such as derivatives of 1,2,6-hexanetriol (reported in Teigelkamp, S., et al., “Branched poly-labelled oligonucleotides: enhanced specificity of fork-shaped biotinylated oligoribonucleotides for antisense affinity selection,” Nucleic Acids Res. (1993) 21:4651-4652), 1,3-diaminopropanol and pentaerythriol (reported in Shchepinov, M., et al., “Oligonucleotide dendrimers: synthesis and use as polylabelled DNA probes,” Nucleic Acids Res. (1997) 25:4447-4454.

### SUMMARY OF THE INVENTION

The inventions described and claimed herein have many attributes and encompass many embodiments including, but not limited to, those set forth in this Summary. The inventions described and claimed herein are not limited to or by the features or embodiments identified in this Summary, which is included for purposes of illustration only and not restriction.

In one aspect the invention is directed to parallel-stranded hairpins, triplexes formed from parallel-stranded hairpins and a target molecule, and the use of these structures as nucleic acid ligands or aptamers for specific target molecules. Although Applicants do not wish to be bound by any particular theory, it is believed that this property is a function of the secondary and three-dimensional structure of the folded parallel duplex and triplexes. Thus, it becomes highly attractive to envision the use of synthetic nucleic acid triplex structures as nucleic acid aptamers for a desired protein target or other molecules.

Parallel-stranded hairpins offer novel secondary and tertiary structures with aptamer properties. Moreover those hairpins, bound to a single stranded DNA or RNA target, generate triplexes with previously unappreciated aptamer characteristics. Targets may include the following but are not limited to microbial organisms such as virus, bacteria, rickettsia and fungi, agents of biological and chemical warfare, proteins, peptides, dysplastic and metastatic cancer cells, autoimmune antibodies and any molecule mediating a pathologic or other process, or present in the body.

One of the aspects of the present invention includes compositions and methods for the preparation of oligonucleotides carrying modified nucleotides including but not limited to 8-aminoadenine, 8-aminoguanine, and 8-aminohypoxanthine, that are connected 3′ to 3′ or 5′ to 5′ (head-to-head or tail-to tail) to a Hoogsteen pyrimidine strand (parallel-stranded hairpins). These parallel-stranded hairpins of the invention are expected to form secondary and/or tertiary structures with aptamer characteristics for specific molecule targets. An addition aspect of the invention includes the synthesis of parallel-stranded hairpins containing, covalently linked to its structure, a DNA or RNA single-strand target, forming a triplex structure. Those triplexes also demonstrate aptamer capabilities. Another embodiment of the invention includes compositions and methods for the preparation of a library of parallel duplexes using a branching phosphoramidite to prepare two asymmetric tracks using standard phosphoramidites.

The invention includes a nucleic acid ligand comprising a parallel-stranded hairpin. In one aspect of the invention, the parallel-stranded hairpin includes a polypurine part and a polypyrimidine connected at their 5′ ends by a linker, or a polypurine part and a polypyrimidine connected at their 3′ ends by a linker. In a further aspect of the invention, the parallel-stranded hairpins comprise at least one modified aminopurine, perhaps an 8-aminopurine. The 8-aminopurine may be, but is not limited to, 8-aminoadenine, 8-aminoguanine, and 8-aminohypoxanthine. The nucleic acid ligand may be an oligonucleotide triplex.

In another aspect of the invention, the nucleic acid ligand includes an attached polypeptide. The polypeptide may be, but is not limited to, a binder polypeptide or a marker polypeptide.

A further aspect of the invention includes a method for preparing a parallel oligonucleotide duplex, including providing a branching phosphoramidite with a first track and a second track, protecting each of the first track and the second track with a protecting group, where the protecting group of the first track is different from the protecting group of the second track, removing the protecting group of the first track and bonding a purine to the first track, replacing a protecting group on the first track, removing the protecting group of the second track and bonding a pyrimidine to the second track, where the pyrimidine is complementary to the purine previously added, then replacing a protecting group on the second track, and repeating those steps to form a parallel oligonucleotide duplex.

The method of forming a parallel-stranded hairpin may include the step of performing a mix and split procedure. The protecting groups may be selected from an acid labile protecting group, a base labile protecting group, a fluoride labile protecting group, a hydrazine labile protecting group, and a photolabile protecting group. The acid labile protecting group may be, but is not limited to, dimethoxytrityl and monomethoxytril. The base labile protecting group may be, but is not limited to, fluorenylmethoxycarbonyl. The fluoride labile protecting group may be, but is not limited to tert-butyldimethylsilyl. The hydrazine labile protecting group may be, but is not limited to, levulenyl. The photolabile protecting group may be, but is not limited to o-Nitrobenzyl. In a still further aspect of the invention, the parallel oligonucleotide duplex includes at least one 8-aminoguanine, 8-aminoadenine, 8-aminohypoxanthine and 5-methylcytosine.

Another aspect of the invention includes a method for binding a target molecule including providing a target molecule, providing a nucleic acid ligand that includes a parallel-stranded hairpin, wherein said parallel-stranded hairpin has a secondary or tertiary structure that allows binding with said target molecule, combining the target molecule and the nucleic acid ligand; and binding the target molecule to the nucleic acid ligand. The parallel-stranded hairpin may include at least one 8-aminopurine, including but not limited to 8-aminoadenine, 8-aminoguanine, and 8-aminohypoxanthine. The nucleic acid ligand may be an oligonucleotide triplex.

The target molecule may be, but is not limited to human proteins, animal proteins, viral proteins, bacterial proteins, peptides, proteins with enzymatic activity, lipases, kinases, esterases, phosphatases, proteases, toxins, prions, hormones, antigen, antibodies, cell receptors, cell surface antigens, adaptor binding proteins, adhesion molecules, apolipoproteins, apoptosis-related proteins, cancer-related proteins, cell cycle proteins, growth factors, Prekallikrein (Fletcher Factor), Kallikrein, Kininogen, Factor I (Fibrinogen), Factor II (Prothrombin), Factor III (Tissue Factor), Factor V, Factor VI (Accelerin), Factor VII, Factor VIII, Factor IX, Factor X, Factor XI (Plasma thromboplastin), Factor XII, Factor XIII, and plasminogen, Plasminogen Activator inhibitor-1 (PAI1), Plasminogen Activator inhibitor-2 (PA2), disease related proteins, cell matrix proteins, cytoskelaton proteins, phosphoproteins, signal transduction related proteins, transcriptional factors, protein translation protein factors, transporters, tissue-specific proteins, complement proteins, carbohydrates, polysaccharides, lipids, Elastase, von Willebrand Factor, Protein C, Protein S, Thrombomodulin, Antithrombin III, Ephreceptor and ephrin-ligand, PTEN, Protein tyrosine phosphatases SHP-1, PRL-3, p53, CDKN2A, MMAC1/PTEN/TEP1 protein, Pim-2, HIV Protease, NS3 protease of hepatitis C virus, Ricin Toxin, 5-alpha reductase, HIV-1 reverse transcriptase, cyclooxygenase, S-adenosylhomocysteine, caffeine, cocaine, and neurotransmitters.

In a further aspect of the invention, the nucleic acid ligand is stable in a human patient. The nucleic acid ligand or a plurality of nucleic acid ligands may be bound to a solid support, for instance for performing an assay. The nucleic acid ligand may be administered to a human patient in need of treatment. The nucleic acid ligand may be administered while bound to a carrier. The carrier may be selected from, but is not limited to, an erythrocyte or an erythrocyte ghost. Once bound to the target, the complex may be eliminated from the body of a patient through the apoptotic cell pathway or through complement activity, though other methods are also possible.

Nucleic acid ligands of the invention may include, but is not limited to, a parallel-stranded hairpin selected from the group consisting of PSH01, PSH02, PSH03, PSH04, PSH05, PSH06, PSH07, PSH08, PSH09, PSH10, PSH11, PSH12, PSH13, PSH14, PSH15, PSH16, PSH17, PSH18, PSH19, PSH20, PSH21, PSH22, PSH23, PSH24, PSH25, PSH26, PSH27, PSH28, PSH29, PSH30, PSH31, PSH32, PSH33, PSH34, PSH35, PSH36, PSH37, PSH38, PSH39, PSH40, PSH41, PSH42, PSH43, PSH44, PSH45, PSH46, PSH47. In a further aspect of the invention, a nucleic acid ligand may include but is not limited to an oligonucleotide triplex. The oligonucleotide triplex may be selected from the group including TS10, TS02, and TS03.

### BRIEF DESCRIPTION OF THE DRAWINGS

FIG. 1 shows hypothetical base-pairing schemes of triads containing 8-aminopurines.

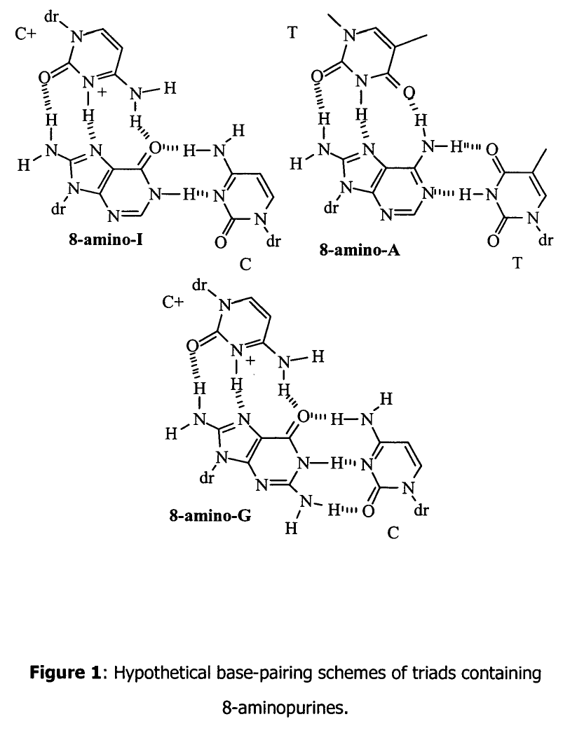

FIG. 2 shows a scheme of binding a polypyrimidine single-stranded nucleic acid with parallel-stranded hairpins.

FIG. 3 shows a simplified scheme for synthesis of a library of parallel duplexes.

### DESCRIPTION OF THE INVENTION

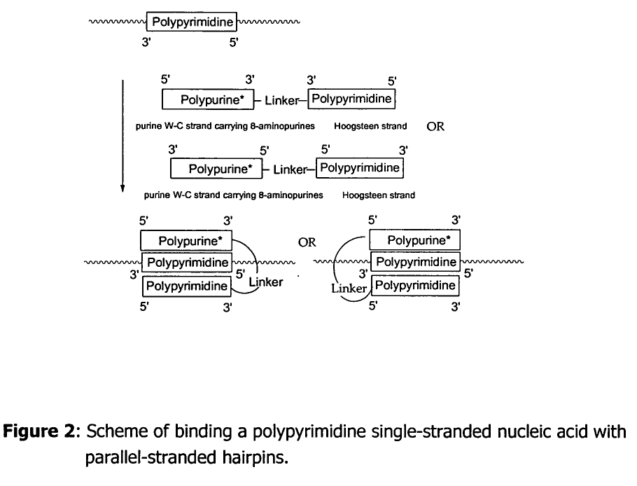

Preferred compositions of the present invention comprise parallel-stranded oligomers having at least one 8-aminopurine. The present invention comprises oligonucleotides derivatives comprising two parts: a polypyrimidine part connected head-to-head to a complementary purine part carrying one or more 8-aminopurines such as 8-aminoadenine, 8-aminoguanine, 8-aminohypoxanthine. A linker molecule is located between both parts in such a way that both parts can form a double stranded structure in parallel sense (FIG. 2). Additionally, a polypyrimidine part may be connected tail-to-tail to a complementary purine part carrying one or more 8-aminopurines. A preferred method for synthesizing the oligonucleotides of the present invention comprises use of phosphoramidite chemistry. Oligonucleotides can be synthesized by any method known to those skilled in the art. Preferred parallel-stranded hairpins of the invention are as long as 40 bases (20 normal polarity and 20 reversed polarity) not including any linking molecule.

Another aspect of the present invention includes methods and compositions for using parallel-stranded hairpins and parallel stranded hairpins bound to a single-stranded target (triplexes), wherein the hairpins may contain 8-aminopurines, as nucleic acid aptamers. These novel aptamers have applications including but not limited to detection of pathological targets, inhibition of proteins with enzymatic activity, and clearance of pathological molecules from the bloodstream, tissue, and other bodily fluids.

Potential targets for nucleic acid ligands of the invention include but are not limited to human proteins, animal proteins, viral proteins, bacterial proteins; peptides; proteins with enzymatic activity (lipases; kinases; esterases; phosphatases; proteases; others); toxins; prions (BSE/TSE); hormones; antigen; antibody; cell receptors; cell surface antigens; adaptor binding proteins; adhesion molecules; apolipoproteins; apoptosis related proteins; cancer related proteins; cell cycle proteins; growth factors; coagulation factor proteins; disease related proteins; cell matrix proteins; cytoskelaton proteins; phosphoproteins; signal transduction related proteins; transcriptional factors; protein translation related proteins; transporters; tissue-specific proteins; immunology related proteins; neurology related proteins; complement proteins; any other proteins; carbohydrate; polysaccharide; lipids; neurotransmitters; or any other kind of molecules.

The target may be present in specific biological location including but not limited to tissues, cells (intracellular and extracellular compartments), and organs. The target may also be present in environmental samples such as water, food, air, soil, and any other source.

The nucleic acid ligands of the invention can be used for detection of targets in both in vivo and in vitro applications. For instance, in one in vitro application the parallel-stranded hairpins and/or triplex structures are attached to a solid support such as Biochips format. Biochips are prepared containing one or more nucleic acid ligands, each nucleic acid ligand capable of binding to a specific target molecule. Biochips could be used to test any kind of sample mixture such as body fluids, water, food, and any other type of samples suspected to have the specific target for the nucleic acid ligands. Nucleic acid ligand-target molecule complex are then detected by any suitable method known to those skilled in the art.

The SELEX protocol used for selection of oligonucleotides ligands from combinatorial nucleic acid libraries is not suitable for generation of a random mixture of parallel-duplex structures because the parallel-stranded hairpins of the invention are best made empirically; for a given position on the hairpin, one should find the complementary base, and the direction of the DNA sequence should be inverted in the middle of the sequence, allowing the formation of a parallel-stranded hairpin. One aspect of the present invention includes the study of parallel-duplex hairpins as aptamers by generating and testing defined sequences individually. This process may be speeded, however, by using DNA-chips technology, in particular DNA-chip synthesizers as Genion (FeBit, Mannheim-Germany). These machines are designed to produce in short-time (one day) 40,000 DNA molecules (in pmol amounts), in a chip using photoactive protecting groups.

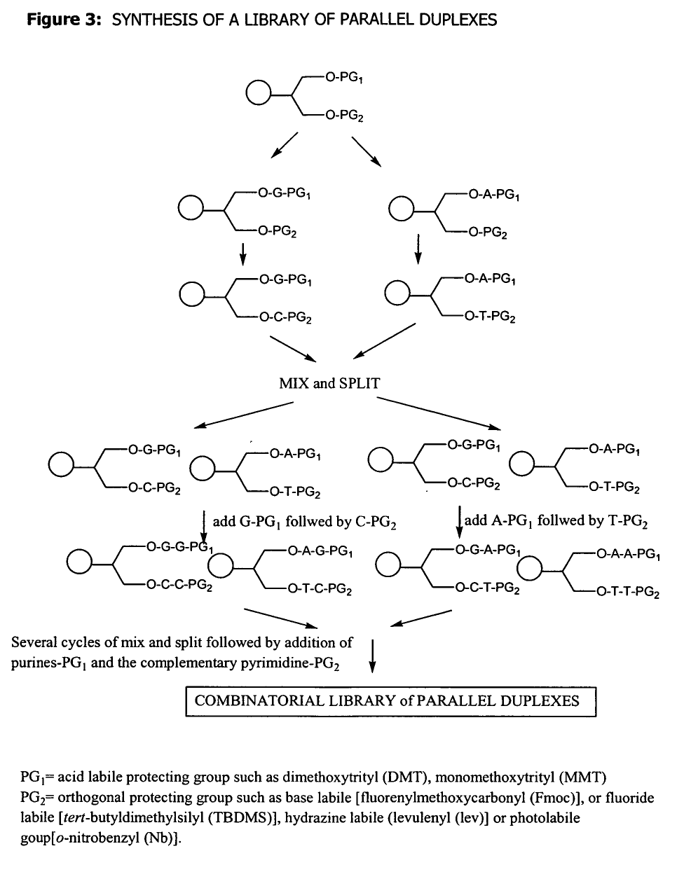

The present invention will also encompass methods and compositions for the synthesis of a library of parallel duplexes as shown, for example, in FIG. 3. In the present invention a branching phosphoramidite is used to prepare two asymmetric tracks using available phosphoramidites (such as those reported in Aviño A., et al., “Synthesis of Branched Oligonucleotides suitable for Triple Helix studies (accepted for publication 2003 Helvetica Chimica Acta)). Branched nucleic acids are prepared using nucleoside and/or non-nucleoside branching molecules. One end of the track (PG₁) is protected by and acid labile group, including but not limited to dimethoxytrityl (DMT) or monomethoxytril (MMT). The other track is protected by a base labile orthogonal group such as but not limited to fluorenylmethoxycarbonyl (Fmoc), or a fluoride labile group such as but not limited to tert-butyldimethysilyl (TBDMS), or a hydrazine labile group sucha as but not limited to levulenyl (Lev), or a photolabile group such as but not limited to o-nitrobenzyl (Nb).

In one aspect of the invention, purine phosphoramidites are prepared with one protector and pyrimidine phosphoramidites with another. Synthesis proceeds by addition of one purine in one of the tracks, followed by removal of the second protecting group and addition of the corresponding complementary pyrimidine. Hairpins containing random sequences on their structure are obtained by a Mix and Split method after a pair of nucleotides have been added (FIG. 3). The order of addition of purines and pyrimidines is not material, so long as they correspond. This strategy provides parallel stranded hairpin structures for analysis as aptamers and ensures that each added purine has a complementary pyrimidine in the parallel strand. This procedure is compatible with the use of non-natural bases including but not limited to 8-aminoguanine, 8-aminoadenine, 5-methylcytosine, among others.

A nonlimiting, exemplary list of parallel-stranded hairpins is included in Table 1. Those skilled in the art will recognize that the linking molecule may be altered in those exemplary hairpins as well. Those sequences are analyzed for binding properties to any known protein, peptide, lipids, and other molecules involved but not limited to pathological disorders. Parallel-duplex hairpin may also contain, covalent linked to its structure, a single DNA or RNA strand target forming a triplex structure. Those triplexes and others according to the invention are evaluated for aptamer capabilities. Moreover, parallel-stranded hairpins and/or triplexes may carry a peptide sequence, which may be used as non-radioactive label; or the peptide can recognize and binds to a specific cell receptor, protein, or other kind of molecule target.

Designed parallel-stranded hairpins or triplex structures have individually selected sequences with the capacity to generate secondary and/or three-dimensional structures that act as a nucleic acid ligands for a desired target molecule. Ideally, candidate structures are able to discriminate between closely related molecules. Nucleic acid ligands may contain modified nucleotides or any kind of chemical modifications to increase the ligand characteristic such as resistance to intra-cellular and extra-cellular nucleases or in vivo stability.

For nucleic acid ligand selection, parallel stranded hairpins and triplex structures are put in contact with a selected target under optimal reaction conditions for binding analysis. The nucleic acid ligand-target pair having the highest affinity and specificity for a desired target molecule is then isolated. The secondary and/or three-dimensional structure of the nucleic acid ligand should be stable under working conditions (in vitro and in vivo) for the nucleic acid ligand to bind its molecule target.

### EXAMPLES

In another embodiment of this invention the nucleic acid ligand can be used in vitro and/or in vivo as inhibitor or activator of a specific target. Potential targets may have enzymatic activity. One example of potential use of nucleic acid ligands of the invention is on patients with respiratory distress syndrome and other lung diseases such as pulmonary emphysema. Due to an overreaction of the immune system white blood cells called neutrophils flock to the lungs, releasing tissue-damaging enzymes such as elastase (as reported in Foronjy, R., et al., “The role of collagenase in emphysema,” Respir Res. (2001) 2:348-352), resulting in the loss of a lung structural protein called Elastin. Parallel-stranded hairpin and triplex structures of the invention are screened for binding capabilities to Elastase. Nucleic acid ligand candidates are useful for therapeutic purposes to prevent the enzyme from degrading connective tissue in the lung.

Another aspect of the invention includes methods and compositions for the in vivo clearance of pathologic and other targets from the peripheral blood and other body fluids. Targets may include but are not limited to dysplastic and metastatic cancer cells. A nucleic acid ligand of the invention that binds to a specific tumor antigen is preferred. Nucleic acid ligands are administrated directly to the body using a suitable carrier for cells-clearance purpose. One such method includes sensitizing red blood cells or ghost erythrocyte cells with the nucleic acid ligands by a suitable covalent or non-covalent method. The method includes administering to patient at least one sensitized erythrocyte or ghost erythrocyte having a nucleic acid ligand capable of binding a target pathological agent and eliminating the bound agents from the patient's blood. Elimination may be achieved through complement proteins or by delivery of the bound complex for destruction through the apoptotic cell pathway by Selected Target Elimination (STE I/STE II), as reported in U.S. Published patent application No. 2003-0232045 A1, in the name of Ramberg, et al. The patient in such a process is a human being, non-human primate, or other animal.

A non-limiting, exemplary list of targets for screening with parallel stranded duplexes and triplexes of the invention include, for example, but are not limited to, blood coagulation targets such as Prekallikrein (Fletcher Factor), Kallikrein, Kininogen, Factor I (Fibrinogen), Factor II (Prothrombin), Factor III (Tissue Factor), Factor V, Factor VI (Accelerin), Factor VII, Factor VIII, Factor IX, Factor X, Factor XI (Plasma thromboplastin), Factor XII, Factor XIII, and plasminogen; plasminogen activators including Plasminogen Activator inhibitor-1 (PAI1), Plasminogen Activator inhibitor-2 (PAI2); regulatory and other proteins including von Willebrand Factor, Protein C, Protein S, Thrombomodulin, Antithrombin III; cancer-related proteins such as Ephreceptor and ephrin-ligand, PTEN, Protein tyrosine phosphatases SHP-1, PRL-3, p53, CDKN2A, MMAC1/PTEN/TEP1 protein, Pim-2; proteases such as Elastase, HIV Protease, NS3 protease of hepatitis C virus; and other proteins and molecules, including Prions (BSE/TSE), Ricin Toxin, 5-alpha reductase, HIV-1 reverse transcriptase, cyclooxygenase, S-adenosylhomocysteine, caffeine, and cocaine.

Patents, patent applications, publications, scientific articles, books, web sites, and other documents and materials referenced or mentioned herein are indicative of the levels of skill of those skilled in the art to which the inventions pertain. Each such referenced document and material is hereby incorporated by reference to the same extent as if it had been incorporated by reference in its entirety individually or set forth or reprinted herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, and the claims referred to above including but not limited to any original claims.

The inventions have been described broadly and generically herein. Each of the narrower species and subgeneric groupings falling within the generic disclosure also form part of these inventions. This includes the generic description of each invention which hereby include, including any claims thereto, a proviso or negative limitation removing or optionally allowing the removal of any subject matter from the genus, regardless of whether or not the excised materials or options were specifically recited or identified in haec verba herein, and all such variations form a part of the original written description of the inventions. In addition, where features or aspects of an invention are described in terms of a Markush group, the invention shall be understood thereby to be described in terms of each and every, and any, individual member or subgroup of members of the Markush group.

The inventions illustratively described and claimed herein can suitably be practiced in the absence of any element or elements, limitation or limitations, not specifically disclosed herein or described herein as essential. Thus, for example, the terms “comprising,” “including,” “containing,” “for example”, etc., shall be read expansively and without limitation. In claiming their inventions, the inventors reserve the right to substitute any transitional phrase with any other transitional phrase, and the inventions shall be understood to include such substituted transitions and form part of the original written description of the inventions. Thus, for example, the term “comprising” may be replaced with either of the transitional phrases “consisting essentially of” or “consisting of.”

As used herein and in the appended claims, the singular forms “a,” “an,” and “the” include plural reference unless the context clearly dictates otherwise.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement was specifically and without qualification or reservation expressly adopted by Applicants in a responsive writing specifically relating to the application that led to this patent prior to its issuance.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Where examples are given, the description shall be construed to include but not to be limited to only those examples. It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

TABLE 1
LIST OF PARALLEL STRANDED HAIRPINS AND NUCLEIC ACID TRIPLEXES

| | | |
|---|---|---|
| Sequence ID PSH01 | ^{5′}GAAGGAGGAGA^{3′}-(EG)₆-^{3′}TCTCCTCCTTC^{5′} | (SEQ ID NO. 1, SEQ ID NO. 2) |
| Sequence ID PSH02 | ^{5′}GAAGGA^{N}GGA^{N}GA^{3′}-(EG)₆-^{3′}TCTCCTCCTTC^{5′} | (SEQ ID NO 3, SEQ ID NO 2) |
| Sequence ID PSH03 | ^{5′}GAAGG^{N}AGG^{N}AGA^{3′}-(EG)₆-^{3′}TCTCCTCCTTC^{5′} | (SEQ ID NO 4, SEQ ID NO 2) |
| Sequence ID PSH04 | ^{5′}GAAGI^{N}AGI^{N}AGA^{3′}-(EG)₆-^{3′}TCTCCTCCTTC^{5′} | (SEQ ID NO 5, SEQ ID NO 2) |
| Sequence ID PSH05 | ^{3′}AGAGGAGGAAG^{5′}-(EG)₆-^{5′}CTTGCTCCTGT^{3′} | (SEQ ID NO 1, SEQ ID NO 2) |
| Sequence ID PSH06 | ^{3′}AGA^{N}GGA^{N}GGAAG^{5′}-(EG)₆-^{5′}CTTCCTCCTCT^{3′} | (SEQ ID NO 3, SEQ ID NO 2) |
| Sequence ID PSH07 | ^{3′}AGAG^{N}GAG^{N}GAAG^{5′}-(EG)₆-^{5′}CTTCCTCCTCT^{3′} | (SEQ ID NO 4, SEQ ID NO 2) |
| Sequence ID PSH08 | ^{3′}AGA^{N}G^{N}GA^{N}G^{N}GAAG^{5′}-(EG)₆-^{5′}CTTCCTCCTCT^{3′} | (SEQ ID NO 6, SEQ ID NO 2) |
| Sequence ID PSH09 | ^{3′}AGA^{N}GGA^{N}GGAAG^{5′}-(EG)₆-^{5′}TTTTTCCCCCC^{3′} | (SEQ ID NO 3, SEQ ID NO 7) |
| Sequence ID PSH10 | ^{3′}AGA^{N}GGA^{N}CGAAG^{5′}-(EG)₆-^{5′}CTTCTTCCTCT^{3′} | (SEQ ID NO 8, SEQ ID NO 9) |
| Sequence ID PSH11 | ^{3′}AGA^{N}GGA^{N}CGAAG^{5′}-(EG)₆-^{5′}CTTCCTCCTCT^{3′} | (SEQ ID NO 8, SEQ ID NO 2) |
| Sequence ID PSH12 | ^{3′}AGA^{N}GGA^{N}CGAAG^{5′}-(EG)₆-^{5′}CTTCGTCCTCT^{3′} | (SEQ ID NO 8, SEQ ID NO 10) |
| Sequence ID PSH13 | ^{3′}AGA^{N}GGA^{N}CGAAG^{5′}-(EG)₆-^{5′}CTTCATCCTCT^{3′} | (SEQ ID NO 8, SEQ ID NO 11) |
| Sequence ID PSH14 | ^{3′}AGA^{N}GGA^{N}CGAAG^{5′}-(EG)₆-^{5′}CTTCpdTCCTCT^{3′} | (SEQ ID NO 8) |
| Sequence ID PSH15 | ^{3′}AGA^{N}GGA^{N}TGAAG^{5′}-(EG)₆-^{5′}CTTCCTCCTCT^{3′} | (SEQ ID NO 12, SEQ ID NO 2) |
| Sequence ID PSH16 | ^{3′}AGA^{N}GGA^{N}TGAAG^{5′}-(EG)₆-^{5′}CTTCTTCCTCT^{3′} | (SEQ ID NO 12, SEQ ID NO 9) |
| Sequence ID PSH17 | ^{3′}AGA^{N}GGA^{N}TGAAG^{5′}-(EG)₆-^{5′}CTTCGTCCTCT^{3′} | (SEQ ID NO 12, SEQ ID NO 10) |
| Sequence ID PSH18 | ^{3′}AGA^{N}GGA^{N}TGAAG^{5′}-(EG)₆-^{5′}CTTCATCCTCT^{3′} | (SEQ ID NO 12, SEQ ID NO 11) |
| Sequence ID PSH19 | ^{3′}AGA^{N}GGA^{N}TGAAG^{5′}-(EG)₆-^{5′}CTTCpdTCCTCT^{3′} | (SEQ ID NO 12) |
| Sequence ID PSH20 | ^{3′}AGA^{N}GGA^{N}GGAAG^{5′}-5′TTTT-CTTCCTCCTCT^{3′} | (SEQ ID NO 3, SEQ ID NO 13) |
| Sequence ID PSH21 | ^{3′}AGA^{N}GGA^{N}GGAAG-TTTT^{5′}-^{5′}CTTCCTCCTCT^{3′} | (SEQ ID NO 14, SEQ ID NO 2) |
| Sequence ID PSH22 | ^{3′}AGA^{N}GGA^{N}GGAAG-GGAGG^{5′}-^{5′}CTTCCTCCTCT^{3′} | (SEQ ID NO 15, SEQ ID NO 2) |
| Sequence ID PSH23 | ^{3′}AGA^{N}GGA^{N}GGAAG-CTTTG^{5′}-^{5′}CTTCCTCCTCT^{3′} | (SEQ ID NO 16, SEQ ID NO 2) |
| Sequence ID PSH24 | ^{3′}AGAGGAGGAAG^{5′}-Naphthalene- | (SEQ ID NO 1, SEQ ID NO 2) |
| | ^{5′}CTTCCTCCTCT^{3′} | |
| Sequence ID PSH25 | 5′-CTCTTTTT-3′-(EG)₆-3′-AAAAAG^{N}AG-5′ | (SEQ ID NO 17, SEQ ID NO 18) |
| Sequence ID PSH26 | 5′-TCCCTCTTTTT-3′-(EG)₆-3′-AAAAAG^{N}AGCGA-5′ | (SEQ ID NO 19, SEQ ID NO 20) |
| Sequence ID PSH27 | 5′-TCTCTTTTTTT-3′-(EG)6-3′-AAAAAACAG^{N}A-5′ | (SEQ ID NO 21, SEQ ID NO 22) |
| Sequence ID PSH28 | 5′-CTCTTTTT-3′-(EG)₆-3′-AAAAAG^{N}AG 5′ | (SEQ ID NO 17, SEQ ID NO 18) |
| | (phosphorothioate linkages) | |
| Sequence ID PSH29 | 5′-GAAGGAGGAGA-TT-3′-bpa- | (SEQ ID NO 23, SEQ ID NO 24) |
| | 3′-TT-TCTCCTCCTTC-5′ | |
| Sequence ID PSH30 | 5′-GAAGGAGGAGA-TT-3′-bppd- | (SEQ ID NO 23, SEQ ID NO 24) |
| | 3′-TT-TCTCCTCCTTC-5′ | |
| Sequence ID PSH31 | 5′-GAAGGA^{N}GGA^{N}GA-TT-3′-bppd- | (SEQ ID NO 25, SEQ ID NO 26) |
| | 3′-UUUCUCCUCCUUC5′ | |
| Sequence ID PSH32 | 5′-GAAGGA^{N}GGA^{N}GA-TT-3′-ppd- | (SEQ ID NO 25, SEQ ID NO 27) |
| | 3′-TMTMMTMMTTM-5′ | |
| Sequence ID PSH33 | 3′CTCCGCTTCCTC-5′-(EG)₆- | (SEQ ID NO 28, SEQ ID NO 29) |
| | 5′-GAG^{N}GAAG^{N}TGG^{N}AGG-hexyl-NH₂-3′ | |
| Sequence ID PSH34 | NH₂-5′-CTTCGCCCCCTTC-3′-(EG)₆- | (SEQ ID NO 30, SEQ ID NO 31) |
| | 3′-GAAGG^{N}GGGTG^{N}AAG-5′ | |
| Sequence ID PSH35 | NH₂-5′TCTCCCTTTTTCT-3′-(EG)₆- | (SEQ ID NO 32, SEQ ID NO 33) |
| | 3′-AGAAAAAAG^{N}GGAG^{N}A-5′ | |
| Sequence ID PSH36 | NH₂-5′TCTCCCTTTTTCT-3′-(EG)₆- | (SEQ ID NO 32, SEQ ID NO 34) |
| | 3′-AGAAAAAAGGGAGA-5′ | |
| Sequence ID PSH37 | 5′-TCTGGGTTTTTCT-TT-(biotin T)-TT- | (SEQ ID NO 35, SEQ ID NO 36) |
| | 3′AGAAAAAGGGAGA-5′ | |
| Sequence ID PSH38 | 5′-TATCCAAGAAAGGA-3′- | (SEQ ID NO 37, SEQ ID NO 38) |
| | 3′-TTTT-TCCTTTCTT-5′-(EG)₄ biotin | |
| Sequence ID PSH39 | NH₂-5′-CCTCCTTTTTCCCGGTC-3′-(EG)₆- | (SEQ ID NO 39, SEQ ID NO 40) |
| | 3′-GA^{N}GGGGGAA^{N}ATAGGA^{N}GG-5′ | |
| Sequence ID PSH40 | 5′-GGAGG^{N}AAGGTG^{N}GGGAC-(EG)₆- | (SEQ ID NO 41, SEQ ID NO 42) |
| | TCCCCGCCTTCCTCC-5′ | |
| Sequence ID PSH41 | 5′-Biotin-GGAAAAAGAAGA-3′-(EG)₆- | (SEQ ID NO 43, SEQ ID NO 44) |
| | 3′-TGTTCTTTTTCC-5′ | |
| Sequence ID PSH42 | 5′-TGCGGAAAAAGAAGA-3′-(EG)₆- | (SEQ ID NO 45, SEQ ID NO 44) |
| | 3′-TCTTCTTTTTCC-biotin-5′ | |
| Sequence ID PSH43 | 5′-CCAACCTTGCGGAAAAAGAAGA-3′-(EG)₆- | (SEQ ID NO 46, SEQ ID NO 44) |
| | 3′-TCTTCTTTTTCC-biotin-5′ | |
| Sequence ID PSH44 | 5′-CCAACCTTGCGGA^{N}AA^{N}AAGA^{N}AGA-3′-(EG)₆- | (SEQ ID NO 47, SEQ ID NO 44) |
| | 3′-TCTTGTTTTTCC-biotin-5′ | |
| Sequence ID PSH45 | 3′-biotin-AGAAGAAGAAGA-5′-(EG)₆- | (SEQ ID NO 48, SEQ ID NO 49) |
| | 5′-TCTTCTTCTTCT-3′ | |
| Sequence ID PSH46 | 3′-ACCTTATTAAATAGAAGAAGAAGA-5′-(EG)₆- | (SEQ ID NO 50, SEQ ID NO 49) |
| | 5′-TCTTCTTCTTCT-biotin-3′ | |
| Sequence ID PSH47 | 3′-ACCTTATTAAATAGAA^{N}GAA^{N}GAA^{N}GA-5′-(EG)₆- | (SEQ ID NO 51, SEQ ID NO 49) |
| | 5′-TGTTGTTCTTCT-biotin-3′ | |
| Sequence ID TS01 | 5′-GAAGGAGGAGA-3′-(EG)₆-bpp-[(EG)₆- | (SEQ ID NO 1, SEQ ID NO 52, SEQ ID NO 2) |
| | 3′-CGTTCCTCCTCT-5′]-(EG)₆-3′-TCTCCTCCTTC-5′ | |
| Sequence ID TS02 | 3′-T₁₃-(EG)₆-A₁₂-5′-(EG)₆-5′-T₁₂-3′ | (SEQ ID NO 53, SEQ ID NO 54, SEQ ID NO 55) |
| Sequence ID TS03 | 3′-T₈-(EG)₆-A₈-5′-(EG)₆-5′-T₈-3′ | (SEQ ID NO 56, SEQ ID NO 57, SEQ ID NO 56) |

Abbreviations: A^{N}=8-amino-adenine; G^{N}=8-amino-guanine; 1^{N}=8-amino-hypoxanthine, (EG)₆ =hexaethyleneglycol, pd=1,3-propanediol, naphthalene=naphthalene derivative described in Bevers et al. (2000) J. Am. Chem. Soc., 122,:5905-5915; bpa: [—PO₃—O(CH₂)₃—CONH—CH₂]₂—CHOH; bppd: [—PO₃—O(CH₂)₄—CONH—CH₂]₂—CHOPO₂OCH₂CH₂CH₂OH; bpp: [—PO₃—O(CH₂)₄—CONH—CH₂]₂—CHOPO₃; M:5-methylcytosine, U,C: 2-O′-methyl-RNA.

1. A nucleic acid ligand comprising a parallel-stranded hairpin.

2. The nucleic acid ligand of claim 1, wherein said parallel-stranded hairpin comprises a polypurine part and a polypyrimidine connected at their 5′ ends by a linker.

3. The nucleic acid ligand of claim 1, wherein said parallel-stranded hairpins comprise a polypurine part and a polypyrimidine connected at their 3′ ends by a linker.

4. The nucleic acid ligand of claim 1, wherein said parallel-stranded hairpins comprise at least one modified aminopurine.

5. The nucleic acid ligand of claim 1, wherein said parallel-stranded hairpins comprise at least one 8-aminopurine.

6. The nucleic acid ligand of claim 5, wherein said 8-aminopurine is selected from the group consisting of 8-aminoadenine, 8-aminoguanine, and 8-aminohypoxanthine.

7. The nucleic acid ligand of claim 1, wherein said nucleic acid ligand is an oligonucleotide triplex.

8. The nucleic acid ligand of claim 7, wherein said oligonucleotide triplex comprises at least one 8-aminopurine.

9. The nucleic acid ligand of claim 1, further comprising a polypeptide.

10. The nucleic acid ligand of claim 9, wherein said polypeptide is a binder polypeptide.

11. The nucleic acid ligand of claim 9, wherein said polypeptide is a label polypeptide.

12. A method for preparing a parallel oligonucleotide duplex, comprising:
(a) providing a branching phosphoramidite with a first track and a second track;
(b) protecting each of said first track and said second track with a protecting group, wherein the protecting group of the first track is different from the protecting group of the second track;
(c) removing the protecting group of the first track and bonding a purine to said first track;
(d) replacing a protecting group on the first track;
(e) removing the protecting group of the second track and bonding a pyrimidine to said second track, wherein said pyrimidine is complementary to the purine of step (c);
(f) replacing a protecting group on the second track; and
(g) repeating steps (c) through (e) to form a parallel oligonucleotide duplex.

13. The method of claim 12, further comprising the step of performing a mix and split procedure before step (g).

14. The method of claim 12, wherein said protecting groups are selected from the group consisting of an acid labile protecting group, a base labile protecting group, a fluoride labile protecting group, a hydrazine labile protecting group, and a photolabile protecting group.

15. The method of claim 14, wherein said acid labile protecting group is selected from the group consisting of dimethoxytrityl and monomethoxytril.

16. The method of claim 14, wherein said base labile protecting group is fluorenylmethoxycarbonyl.

17. The method of claim 14, wherein said fluoride labile protecting group is tert-butyldimethylsilyl.

18. The method of claim 14, wherein said hydrazine labile protecting group is levulenyl.

19. The method of claim 14, wherein said photolabile protecting group is o-Nitrobenzyl.

20. The method of claim 14, wherein said parallel oligonucleotide duplex comprises at least member of the group consisting of 8-aminoguanine, 8-aminoadenine, 8-aminohypoxanthine and 5-methylcytosine.

21. A method for binding a target molecule comprising:
(a) providing a target molecule;
(b) providing a nucleic acid ligand comprising a parallel-stranded hairpin, wherein said parallel-stranded hairpin has a secondary or tertiary structure that allows binding with said target molecule;
(c) combining said target molecule and said nucleic acid ligand; and
(d) binding said target molecule to said nucleic acid ligand.

22. The method of claim 21, including wherein said parallel-stranded hairpin includes at least one 8-aminopurine.

23. The method of claim 22, including wherein said at least one 8-aminopurine is selected from the group consisting of 8-aminoadenine, 8-aminoguanine, and 8-aminohypoxanthine.

24. The method of claim 21, including wherein said nucleic acid ligand is an oligonucleotide triplex

25. The method of claim 21, including wherein said target molecule is selected from the group consisting of human proteins, animal proteins, viral proteins, bacterial proteins, peptides, proteins with enzymatic activity, lipases, kinases, esterases, phosphatases, proteases, toxins, prions, hormones, antigen, antibodies, cell receptors, cell surface antigens, adaptor binding proteins, adhesion molecules, apolipoproteins, apoptosis-related proteins, cancer-related proteins, cell cycle proteins, growth factors, Prekallikrein (Fletcher Factor), Kallikrein, Kininogen, Factor I (Fibrinogen), Factor II (Prothrombin), Factor II (Tissue Factor), Factor V, Factor VI (Accelerin), Factor VII, Factor VIII, Factor IX, Factor X, Factor XI (Plasma thromboplastin), Factor XII, Factor XIII, and plasminogen, Plasminogen Activator inhibitor-1 (PAI1), Plasminogen Activator inhibitor-2 (PAI2), disease related proteins, cell matrix proteins, cytoskelaton proteins, phosphoproteins, signal transduction related proteins, transcriptional factors, protein translation protein factors, transporters, tissue-specific proteins, complement proteins, carbohydrates, polysaccharides, lipids, Elastase, von Willebrand Factor, Protein C, Protein S, Thrombomodulin, Antithrombin III, Eph-receptor and ephrin-ligand, PTEN, Protein tyrosine phosphatases SHP-1, PRL-3, p53, CDKN2A, MMAC1/PTEN/TEP1 protein, Pim-2, HIV Protease, NS3 protease of hepatitis C virus, Ricin Toxin, 5-alpha reductase, HIV-1 reverse transcriptase, cyclooxygenase, S-adenosylhomocysteine, caffeine, cocaine, and neurotransmitters.

26. The method of claim 21, including wherein said nucleic acid ligand is stable in a human patient.

27. The method of claim 21, including wherein said nucleic acid ligand is bound to a solid support.

28. The method of claim 21, including wherein said nucleic acid ligand is administered to a human patient in need of treatment.

29. The method of claim 28, including wherein said nucleic acid ligand is administered while bound to a carrier.

30. The method of claim 29, including wherein said carrier is selected from the group consisting of an erythrocyte and an erythrocyte ghost.

31. The method of claim 30, including the further step of eliminating said nucleic acid ligand, target, and carrier from the body of a patient through the apoptotic cell pathway.

32. The method of claim 30, including the further step of eliminating said nucleic acid ligand, target, and carrier from the body of a patient through complement activity.

33. A nucleic acid ligand comprising a parallel-stranded hairpin, wherein said parallel-stranded hairpin is selected from the group consisting of PSH01, PSH02, PSH03, PSH04, PSH05, PSH06, PSH07, PSH08, PSH09, PSH10, PSH11, PSH12, PSH13, PSH14, PSH15, PSH16, PSH17, PSH18, PSH19, PSH20, PSH21, PSH22, PSH23, PSH24, PSH25, PSH26, PSH27, PSH28, PSH29, PSH30, PSH31, PSH32, PSH33, PSH34, PSH35, PSH36, PSH37, PSH38, PSH39, PSH40, PSH41, PSH42, PSH43, PSH44, PSH45, PSH46, PSH47.

34. A nucleic acid ligand including an oligonucleotide triplex, said oligonucleotide triplex selected from the group consisting of TS01, TS02, and TS03.

35. Parallel-stranded hairpin sequences as aptamers.

36. Oligonucleotide triplexes as aptamers.
